(19) United States
(12) Patent Application Publication
(10) Pub. No.: US 2011/0295070 A1
(43) Pub. Date: Dec. 1, 2011

(54) ENDOSCOPE APPARATUS

(75) Inventor: Shinji YASUNAGA, Asaka-shi (JP)

(73) Assignee: OLYMPUS CORPORATION, Tokyo (JP)

(21) Appl. No.: 13/114,231

(22) Filed: May 24, 2011

(30) Foreign Application Priority Data
May 26, 2010 (JP) JP 2010-120493

(51) Int. Cl. A61B 1/12

(52) U.S. Cl. 600/157

(57) ABSTRACT

Provided is an endoscope apparatus which is equipped with an endoscope including an object lens, a distal end portion and a handling section, a heat-producing device being incorporated into the distal end portion. The endoscope apparatus includes: a cleaning system which transiently discharges a cleaning liquid toward the object lens to clean the object lens; a cooling system which refluxes a coolant in the distal end portion to cool the heat-producing device; a liquid sending system, installed outside the endoscope, for supplying the cleaning liquid and the coolant; and a liquid-sending pressurizing system shared between the cleaning liquid and the coolant.

### CROSS-REFERENCE TO RELATED APPLICATION

The present application is based upon and claims the benefit of priority from the prior Japanese Patent Application No. 2010-120493 filed on May 26, 2010; the entire contents of which are incorporated herein by reference.

### BACKGROUND OF THE INVENTION

1. Field of the Invention

The present invention relates to an endoscope apparatus.

2. Description of the Related Art

In association with sophistication in the capabilities of endoscope apparatuses, the quantity of heat produced at the distal end of an endoscope is on the increase. Accordingly, a technique of refluxing fluid in the distal end to provide cooling for the same has been desired. An endoscope apparatus equipped with such a distal-end cooling system is disclosed in, e.g., Japanese Published Patent Application Publication No. 2007-7322.

The endoscope apparatus disclosed in Japanese Published Patent Application Publication No. 2007-7322 will be hereinafter discussed with reference to FIG. 2. FIG. 2 shows a configuration of a conventional endoscope apparatus. In the endoscope apparatus shown in FIG. 2, tubes (supply and return channels) through which a coolant circulates at all times are installed in an endoscope insertion portion 901. The coolant circulates through these tubes via a pump 922 that is installed in a handling section 921 to thereby provide cooling for an LED installed in an distal end body 903. In addition, the handling section 921 is connected to a power supply 925 via a universal cord 923. The power supply 925 is connected to a monitor 929 via a video processor 927 and also to a suction bottle 932 via a suction pump 931.

However, in the above-described conventional endoscope apparatus, the pump 922 is newly incorporated into the handling section 921 of the endoscope, which has been a cause of an increase in production cost, when compared with an endoscope apparatus having no cooling system at the distal end.

### SUMMARY OF THE INVENTION

The present invention has been made in view of the foregoing circumstances, and an object of the present invention is to provide an endoscope apparatus which is devised to be capable of minimizing the increase in production cost though having a distal-end cooling system.

To solve the above-described problem and achieve the object mentioned above, according to the present invention, there is provided an endoscope apparatus which is equipped with an endoscope including an object lens, a distal end portion and a handling section, a heat-producing device being incorporated into the distal end portion. The endoscope apparatus includes a cleaning system which transiently discharges a cleaning liquid toward the object lens to clean the object lens, and a cooling system which refluxes a coolant in the distal end portion to cool the heat-producing device. The endoscope apparatus further includes a liquid sending system, installed outside the endoscope, for supplying the cleaning liquid and the coolant, and a liquid-sending pressurizing system shared between the cleaning liquid and the coolant.

In the endoscope apparatus according to the present invention, it is desirable that the cleaning liquid and the coolant be supplied to the endoscope from the liquid sending system through a common liquid supply tube.

In the endoscope apparatus according to the present invention, it is desirable that liquid supplied to the endoscope through the common liquid supply tube be bifurcated inside the endoscope to flow into a cleaning liquid sending tube and a coolant sending tube through which the cleaning liquid and the coolant are delivered to the distal end portion, respectively, wherein the cleaning liquid is transiently discharged as needed via a valve installed in the handling section, and wherein the coolant is sent to the distal end portion at all times while the endoscope apparatus is operated.

In the endoscope apparatus according to the present invention, it is desirable that the endoscope apparatus performs one of the following two operations when the cleaning liquid is discharged: temporarily stopping delivery of the coolant; and decreasing a flow rate of the delivery of the coolant.

In the endoscope apparatus according to the present invention, it is desirable that the endoscope apparatus temporarily suppress temperature rise of the heat-producing device when the cleaning liquid is discharged.

In the endoscope apparatus according to the present invention, it is desirable that the endoscope apparatus temporarily suppress the temperature rise of the heat-producing device by a reduction in power supplied to the heat-producing device.

In the endoscope apparatus according to the present invention, it is desirable that a liquid sending capability of the liquid sending system be temporarily increased when the cleaning liquid is discharged.

### BRIEF DESCRIPTION OF THE DRAWINGS

FIG. 1 is a schematic diagram showing the internal structure of a first embodiment of an endoscope apparatus according to the present invention; and

FIG. 2 is a schematic diagram showing the configuration of a conventional endoscope apparatus.

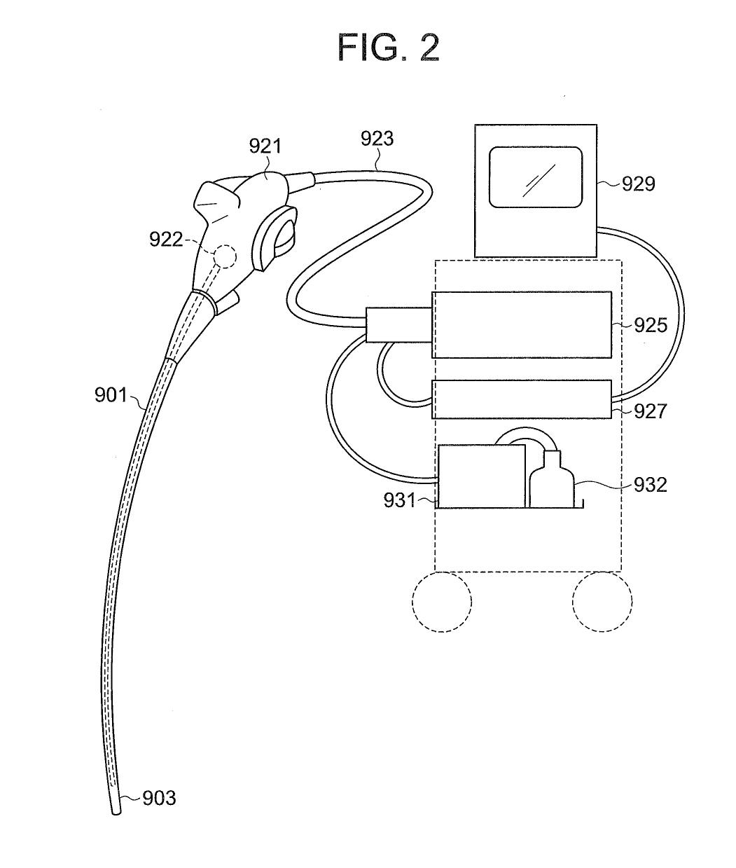

### DETAILED DESCRIPTION OF THE INVENTION

Embodiments of an endoscope apparatus according to the present invention will be discussed below in detail with reference to the accompanying drawings. It should be noted that the present invention is not limited by the specific embodiments described herein.

### First Embodiment

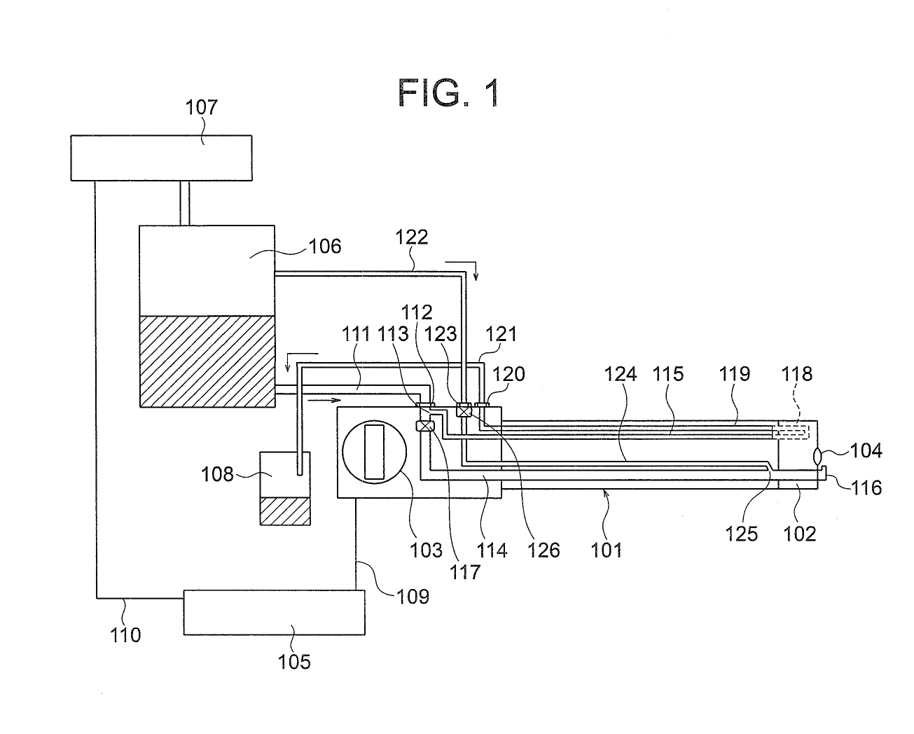

A first embodiment of the endoscope apparatus will be hereinafter discussed with reference to FIG. 1. FIG. 1 schematically shows the structure of the first embodiment of the endoscope apparatus.

An endoscope 101 of the endoscope apparatus is provided, at a distal end side and a proximal end side, with a distal end portion 102 and a handling section 103, respectively. The endoscope 101 is provided inside the distal end portion 102 with an object lens 104 and heat-producing electronic components (heat-producing devices) not shown in the drawings such as an image pickup unit and an illumination LED (light-emitting diode). The endoscope apparatus is provided outside the endoscope 101 with a controller 105, a water tank 106, a compressor 107 (liquid sending system, pressurizing system) and a drain tank 108. The water tank 106 contains a cleaning liquid and a coolant, the compressor 107 operates to send the cleaning liquid and the coolant to the endoscope 101 by applying pressure to the water tank 106, and the drain tank 108 holds drainage discharged from the endoscope 101.

Although water is used as a cleaning liquid and a coolant in the first embodiment of the endoscope apparatus, any other liquid can be used instead.

The controller 105 and the endoscope 101 are electrically connected to each other via a bundle of power/signal transmission cables 109. The bundle of power/signal transmission cables 109 is connected to the handling section 103 of the endoscope 101. The controller 105 is connected to the heat-producing electronic components, which are installed inside the distal end portion 102, via electrical wiring (not shown) installed inside the endoscope 101. In addition, the controller 105 and the compressor 107 are electrically connected to each other via a bundle of power/signal transmission cables 110.

The endoscope apparatus is provided with a common liquid supply tube 111 through which both the cleaning liquid and the coolant flow. The common liquid supply tube 111 is drawn out from the water tank 106 to be connected to the endoscope 101 via a liquid supply tube connector 112 provided on the handling section 103. In the interior of the handling section 103, the common liquid supply tube 111 that is connected to the endoscope 101 via the liquid supply tube connector 112 is bifurcated at a branch portion 113 into two liquid sending tubes: a cleaning liquid sending tube 114 and a coolant sending tube 115.

The cleaning liquid sending tube 114 is connected to a cleaning nozzle 116 at the distal end portion 102. A valve 117 is installed in a portion of the cleaning liquid sending tube 114 in the handling section 103. The valve 117 is opened and closed by operating a valve switch (not shown) provided on the handling section 103.

The coolant sending tube 115 is connected to one end of a flow channel 118 provided in the distal end portion 102, and the other end of the flow channel 118 is connected to the distal end of a coolant discharge tube 119. The coolant discharge tube 119 is installed in the endoscope 101 to extend therethrough toward the handling section 103, and the proximal end of the coolant discharge tube 119 is connected to one end of an external coolant discharge tube 121 via a connector 120 provided on the handling section 103. The external coolant discharge tube 121 is connected at the other end thereof to the drain tank 108.

The endoscope apparatus is provided with an external air supply tube 122 which is connected to the water tank 106 in addition to the common liquid supply tube 111. In the case where the liquid contained in the water tank 106 is pressurized downwards from above by the compressor 107 as shown in FIG. 1, the common liquid supply tube 111 is connected to the bottom of the water tank 106 or a portion of the water tank 106 in the vicinity of the bottom thereof, and an end of the external air supply tube 122 is connected to the top of the water tank 160 or a portion of the water tank 160 in the vicinity of the top thereof.

The other end of the external air supply tube 122 is connected to a connector 123 provided on the handling section 103, thereby being connected to an internal air supply tube 124. The internal air supply tube 124 is installed in the endoscope 101 to extend therethrough toward the distal end portion 102. In addition, the internal air supply tube 124 is coupled (joined) to the cleaning liquid sending tube 114 at a joint 125 formed in the vicinity of the distal end portion 102. In addition, a valve 126 is installed in a portion of the internal air supply tube 124 in the handling section 103. The valve 126 is opened and closed by operating a valve switch (not shown) provided on the handling section 103.

Operations of a cooling system of the endoscope apparatus for cooling the aforementioned heat-producing electronic components, which are installed inside the distal end portion 102, will be discussed hereinafter.

When the endoscope apparatus enters an operating state, the electronic components inside the distal end portion 102 enter operating states thereof and the compressor 107 starts operating. This causes water in the water tank to flow into the flow channel 118 through the common liquid supply tube 111 and the coolant sending tube 115 to thereby provide cooling for the distal end portion 102. The cooling water as a coolant flown into the flow channel 118 in the distal end portion 102 is discharged into the drain tank 108 through the coolant discharge tube 119 and the external coolant discharge tube 121. The flow of the coolant into the flow channel 118 in this way provides cooling for the entirety of the distal end portion 102, which makes it possible to suppress temperature rise of the heat-producing electronic components installed in the distal end portion 102. Here, if the endoscope is in a viewing state other than a state of cleaning the object lens 104, the valve 117 is in the closed state and thus water in the water tank 106 flows solely into the coolant sending tube 115, so that no water drains out of the cleaning nozzle 116 through the cleaning liquid sending tube 114.

Next, operations of a cleaning system for cleaning the object lens 104, and operations of the cooling system during the operation of the cleaning system will be discussed hereinafter.

When mucus on the observation site or the like causes a smear on the object lens 104 during observation, the endoscope operator operates a valve switch (not shown) to open the valve 117. With this opening of the valve 117, water in the water tank 106 is discharged therefrom not only toward the coolant sending tube 115 but also toward the object lens 104 from the tip of the cleaning nozzle 116 via the cleaning liquid sending tube 114, so that the water thus discharged from the cleaning nozzle 116 cleans the object lens 104. This lens cleaning time is in the order of a few seconds.

The change of the state of the valve 117 to the open state is communicated to the controller 105 via the bundle of power/signal transmission cables 109 during the cleaning operation for cleaning the object lens 104. Thereupon, the controller 105 reduces the power supplied to one or more than one of the heat-producing electronic components incorporated into the distal end portion 102, e.g., to the aforementioned illumination LED.

In addition, upon completion of the operation for cleaning the object lens 104, the controller 105 closes the valve 117 and sets the level of the power supplied to the aforementioned heat-producing electronic component or components, which is incorporated into the distal end portion 102, back to the original setting.

During the operation for cleaning the object lens 104, a cleaning liquid is discharged from the cleaning nozzle 116 with the valve 117 open; therefore, the pressure for delivering a cleaning liquid toward the coolant sending tube 115 drops at the branch portion 113, so that the flow rate of the coolant flown into the flow channel 118 reduces. This reduction may cause a decrease in the capability of cooling the distal end portion 102. However, as described above, the temperature rise of the distal end portion 102 can be suppressed by a reduction in power supplied to the heat-producing electronic component or components incorporated into the distal end portion 102.

At this time, in the first embodiment of the endoscope apparatus, the quantity of reduction in power supplied to, e.g., the illumination LED can be set to a level not presenting an obstacle to an observation operation during the object-lens cleaning operation in a normal endoscopic examination or treatment even if the illuminance of the observation field of view decreases.

As described above, in the first embodiment of the endoscope apparatus, both the capability of cleaning the object lens 104 and the capability of cooling the distal end portion 102 can be achieved with a simple configuration by supplying a coolant and a cleaning liquid via the common liquid supply tube 111 and the compressor 107 that are shared between the cooling system and the cleaning system. In addition, control of the power supplied to the heat-producing electronic component or components can suppress temperature rise of the distal end portion 102 that accompanies a reduction in the flow rate of the coolant during the discharge of the cleaning liquid. Accordingly, it is no longer required to install two independent compressors or pumps for supplies of the cooling liquid and the coolant, respectively. Additionally, to ensure a sufficient flow rate for both a coolant and a cleaning liquid at a time, a high-pressure compressor is usually required and tubes, pipes and others to be installed are usually required to be resistant to high pressure and increased in size; however, in the first embodiment of the endoscope apparatus, no high-pressure compressor is required, which makes it possible to prevent the system from becoming complicated and increasing in size.

Modified embodiments of the first embodiment of the endoscope apparatus will be discussed hereinafter.

The valve 117 can be replaced by a valve for switching between a cleaning liquid and a coolant to increase the discharge pressure of the cleaning liquid by shutting off the flow of the coolant with the valve during discharge of the cleaning liquid. Alternatively, during discharge of the cleaning liquid, power supply can be reduced by a reduction in frame rate of the image pickup unit, not by a reduction in quantity of the power supplied to the illumination LED.

In addition, an arrangement in which a coolant tank and a cleaning liquid tank that are independent of each other are pressurized by a single compressor is possible. This arrangement is suitable for the case where it is desirable to use different types of liquids as a cleaning liquid and a coolant. In this case, however, the cleaning liquid and the coolant are supplied to the endoscope through independent liquid sending tubes.

Additionally, although the branch portion 113 and the valve 117 are installed in the handling section 103 in the first embodiment of the endoscope apparatus, the present invention is not limited to this particular installation; for instance, each of the branch portion 113 and the valve 117 can be installed in any part of the endoscope 101. In addition, it is possible that the branch portion 113 and the valve 117 be installed in the controller 105 so that liquid sending tubes (branch tubes) extending from the branch portion 113 are connected to the endoscope 101.

Additionally, the valve switches of the valves 117 and 126 can be installed in any part other than the handling section 103. Additionally, the valves 117 and 126 can be operated not only by manually operating the valve switches by an endoscope operator but also automatically in response to a detection of the adherence of a smear on the object lens 104.

Additionally, although a reduction in power supply to one or more heat-producing electronic components such as the illumination LED is implemented as a measure to reduce the quantity of heat produced at the distal end portion 102 during the operation for cleaning the object lens 104 in the first embodiment of the endoscope apparatus, another measure can be implemented. For instance, it is conceivable, e.g., to reduce or stop the operating frequency or applied voltage of the image pickup unit or peripheral circuits incorporated into the distal end portion 102.

### Second Embodiment

A second embodiment of the endoscope apparatus is identical in structure to the first embodiment of the endoscope apparatus except that the flow rate of a cleaning liquid is ensured with no reduction in the flow rate of a coolant by temporarily increasing the pressure of the compressor 107 when the valve 117 moves to the open state.

The controller of the second embodiment of the endoscope apparatus controls the operation of the compressor 107 in a manner to increase the pressure to the water tank 106 upon detecting that the valve 117 moves to the open state. This control makes it possible to supply the same quantity of coolant as in the closed state of the valve 117 to the flow channel 118 through the coolant sending tube 115 even when the valve 117 moves to the open state.

In this endoscope apparatus, the compressor needs an extra capacity, devices of the endoscope apparatus that are installed outside the endoscope increase in size, and an improvement in resistance to pressure is required for the tubes and pipes installed in the endoscope; however, there is a merit of not complicating the electrical control system since it is not required to control the power supplied to the electronic components installed inside the distal end portion 102 in accordance with changes in the open/closed states of the valve 117.

The other configurations, operations and effects are identical to those in the first embodiment of the endoscope apparatus.

As can be understood from the foregoing, the endoscope apparatus according to the present invention is suitably used as an endoscope apparatus the distal end of which easily produces heat due to sophistication in the capabilities of the endoscope.

The endoscope apparatus according to the present invention offers an effect capable of minimizing the increase in production cost though having a cooling system at the endoscope distal end.

What is claimed is:

1. An endoscope apparatus equipped with an endoscope including an object lens, a distal end portion and a handling section, a heat-producing device being incorporated into the distal end portion, the endoscope apparatus comprising:
a cleaning system which transiently discharges a cleaning liquid toward the object lens to clean the object lens;
a cooling system which refluxes a coolant in the distal end portion to cool the heat-producing device;
a liquid sending system, installed outside the endoscope, for supplying the cleaning liquid and the coolant; and
a liquid-sending pressurizing system shared between the cleaning liquid and the coolant.

2. The endoscope apparatus according to claim 1, wherein the cleaning liquid and the coolant are supplied to the endoscope from the liquid sending system through a common liquid supply tube.

3. The endoscope apparatus according to claim 2, wherein liquid supplied to the endoscope through the common liquid supply tube is bifurcated inside the endoscope to flow into a cleaning liquid sending tube and a coolant sending tube through which the cleaning liquid and the coolant are delivered to the distal end portion, respectively,
wherein the cleaning liquid is transiently discharged as needed via a valve installed in the handling section, and
wherein the coolant is sent to the distal end portion at all times while the endoscope apparatus is operated.

4. The endoscope apparatus according to claim 1, wherein, when the cleaning liquid is discharged, the endoscope apparatus performs one of the following two operations:
temporarily stopping delivery of the coolant; and
decreasing a flow rate of the delivery of the coolant.

5. The endoscope apparatus according to claim 1, wherein, when the cleaning liquid is discharged, the endoscope apparatus temporarily suppresses temperature rise of the heat-producing device.

6. The endoscope apparatus according to claim 5, wherein the endoscope apparatus temporarily suppresses the temperature rise of the heat-producing device by a reduction in power supplied to the heat-producing device.

7. The endoscope apparatus according to claim 1, wherein, when the cleaning liquid is discharged, a liquid sending capability of the liquid sending system is temporarily increased.
